(19) United States
(12) Patent Application Publication
(10) Pub. No.: US 2009/0292343 A1
(43) Pub. Date: Nov. 26, 2009

(54) GARMENT WITH ATTACHABLE ACCESSORY

(76) Inventor: David Sternlight, Canoga Park, CA (US)

Correspondence Address: QUICKPATENTS, INC., 32861 CALLE PERFECTO, SUITE A, SAN JUAN CAPISTRANO, CA, 92675, US

(21) Appl. No.: 12/469,590

(22) Filed: May 20, 2009

Related U.S. Application Data
(60) Provisional application No. 61/055,461, filed on May 23, 2008.
(60) Provisional application No. 61/056,149, filed on May 27, 2008.

(51) Int. Cl. A61F 7/02; A01K 13/00; A61F 7/00

(52) U.S. Cl. 607/112; 119/850; 607/114

(57) ABSTRACT

A garment for a mammal, such as a canine, is disclosed having a flexible web with a first part of a two-part hook-and-loop type fastener on an outer surface, and optionally on an inner surface, thereof. The flexible web includes at least one tab at a peripheral edge thereof that each include a second part of the two-part fastener on an inner surface thereof, and optionally on an outer surface thereof. The flexible web may further including at least one aperture therethrough for receiving a member of the mammal therethrough. An accessory is selectively attachable to the web and includes at least one of the second part of the two-part fastener. The accessory may include a thermal pack adapted to radiate heat to or absorb heat of the mammal at the desired location thereon, or to lower the temperature of the mammal proximate the desired location thereon. The accessory may take the form of a plush animal, for example, which includes at least one selectively closable pocket for containing the thermal pack, the aromatic substance, or other items such as water, food, treats, or the like.

### CROSS-REFERENCE TO RELATED APPLICATIONS

This application claims the benefit of U.S. Provisional Patent Application 61/055,461, filed on May 23, 2008, and U.S. Provisional Patent Application 61/056,149, filed on May 27, 2008, both incorporated herein by reference.

### STATEMENT REGARDING FEDERALLY SPONSORED RESEARCH AND DEVELOPMENT

Not Applicable.

### FIELD OF THE INVENTION

This invention relates to garments, and more particularly to a garment with an attachable accessory.

### DISCUSSION OF RELATED ART

Applying temperature-based therapies to mammals, such as canines, human beings and particularly children, and the like, can be challenging, particularly for active such mammals. Keeping a hot pack or cold pack in place as the animal moves, or in place in an awkward position, for any length of time can be irritating to the animal. For such therapies to be effective, sometimes uninterrupted and extended durations of the therapy must be applied. When the animal is uncooperative, in some cases the animal must be forcefully restrained to effect such therapies.

Several prior art devices are directed at the application of heat-based therapies to mammals. For example, U.S. Pat. No. 6,699,271 to Clayton on Mar. 2, 2004, teaches a therapeutic wrap designed for use principally around the neck and shoulder areas of a human. Such a device can be heated and applied to the neck and shoulder areas of the person for therapy and relaxation. However, such a device is not firmly fixable to the person, and as such is not well suited for placement elsewhere on the person, or on other mammals, as such a device would quickly fall off with little movement.

Another device, taught in U.S. Pat. No. 6,240,882 to Gross on Jun. 5, 2001, teaches a wrap for a four-legged mammal that provides heat to one of the mammal's legs. Such a device may be well-suited for its intended use, but such a device is not well-suited for applying heat therapy to other areas of the mammal, such as the back or neck. Further, such a device is not well suited for carrying additional items, such as while the mammal is on a hike with its owner, or otherwise out in public, for example.

U.S. Pat. No. 5,823,984 to Silverberg on Oct. 20, 1998, teaches an expandable wrap with multiple panels for adapting to fit a wide variety of locations on a person's body.

Attachable pockets for holding a heat or cold pack are included for attaching to an inside surface of the wrap. The panels include insulating rubber portions. Such a device is not suited for attaching the heat packs to the outside surface of the wrap, and as such is more difficult to position and use on the mammal or person. This device teaches away from externally-mounted pockets and thermal packs.

U.S. Pat. No. 7,243,509 to Trinh et al. on Jul. 17, 2007, teaches an apparatus and method for attaching a flexible pocket to a garment at a location on a body proximate an injury. The flexible pocket retains alternating hot and cold packs for applying thermal therapy proximate the injury. Such a pocket is fixed to the garment with either an adhesive or a plurality of safety pins. Adhesives, when applied to a conventional textile garment, quickly lose their effectiveness due to the adhesive becoming soiled by stray fibers from the garment, dust and other debris. Safety pins, however, are time consuming to use and carry with them the significant risk of puncturing the body proximate the injury and causing significant pain and possible further injury. Further, the pocket of such a device is open-ended and as such the thermal pack is prone to becoming dislodged from the pocket when the body is in motion.

Therefore, there is a need for a garment that allows a thermal pack, or other accessory, to be fixed to a person or animal at a wide variety of locations for the purpose of delivering thermal therapy thereto, either on an inside or outside surface of the garment as needed. Such a needed device would be aesthetically pleasing by including only a minimum number of attachment straps and other attaching hardware, having the ability to be reversible so as to provide aesthetic options to the user, and providing a disguised thermal pack carrier in the form of a plush toy, or the like. Such a needed garment could be easily used without the therapy pack carrier or accessory, and could also be used with other accessories such as travel pouches, audio equipment carriers, or the like. Further, the needed garment would also be easy to put on, use, and take off, and would minimize risk of further injury to the mammal to which it is applied. The present invention accomplishes these objectives.

### SUMMARY OF THE INVENTION

The present device is a garment for a mammal, such as a canine. A flexible web has a first part of a two-part fastening means on an outer surface, and optionally an inner surface, thereof. The flexible web includes at least one tab at a peripheral edge thereof that each include a second part of the two-part fastening means on an inner surface thereof, and optionally on an outer surface thereof. One portion of the peripheral edge of the flexible web may include a plush fabric material. The garment may be reversible, and can be applied to the mammal either with the outer surface facing away from the mammal, or with the inner surface facing away from the mammal.

Preferably the two-part fastening means is a hook-and-loop type fastening means. As such, the first part may be the loop part of the hook-and-loop type fastening means, and the second part may be the hook part of the hook and loop type fastening means, or vice versa.

An accessory is selectively attachable to the web and includes at least one of the second part of the two-part fastening means. The accessory may include a thermal pack, for example, adapted to radiate heat through the flexible web to the mammal at the desired location thereon, or to lower the temperature of the mammal proximate the desired location thereon. An aromatic substance such as dried lavender may also be included for producing a pleasant scent when heated.

The accessory may take the form of a plush animal, for example, which includes at least one selectively closable pocket for containing the thermal pack, the aromatic substance, or other items such as water, food, treats, or the like. In such an embodiment, the plush animal may further include a pliable endoskeleton therein, made from a pliable metal framework for example, such that the posturing of the plush animal may be selectively configured. Other types of accessories may be used, such as MP3 player carriers, travel pouches, and the like.

When used with mammals having members such as legs, arms, tails, and heads, such as with canines, the flexible web may further including at least one aperture therethrough for receiving the member of the mammal therethrough. The web may take the form of a jacket for canines, for example, which includes two of the apertures therethrough for the front legs of the canine.

In use, the flexible web is wrapped around the mammal and fixed thereto with the two-part fastening means. The accessory may be attached to the outer surface of the flexible web at any desired location thereon, and, in the embodiment wherein the accessory takes the form of a plush animal with an inner pliable endoskeleton, the plush animal may be selectively postured. In the embodiment wherein the first part of the two-part fastening means is included on the inner surface of the flexible web, the accessory may be fixed to the inner surface of the web, between the web and the mammal.

The present invention is a garment that allows a thermal pack, or other accessory, to be fixed to a person or animal at a wide variety of locations for the purpose of delivering thermal therapy thereto, either on an inside or outside surface of the garment as needed. The present garment is aesthetically pleasing as it includes only a minimum number of attachment tabs, is reversible so as to provide aesthetic options to the user, and can provide a disguised thermal pack carrier in the form of a plush toy, or the like. The present invention is easily used without the therapy pack carrier or accessory, solely as a garment, for example, but can also be used with other accessories such as travel pouches, audio equipment carriers, battery-operated vibrating massage devices, or the like. Further, the present garment is easy to put on, use, and take off, and minimizes risk of further injury to the mammal to which it is applied. Other features and advantages of the present invention will become apparent from the following more detailed description, taken in conjunction with the accompanying drawings, which illustrate, by way of example, the principles of the invention.

### DESCRIPTION OF THE DRAWINGS

FIG. 1 is a perspective view of the invention, illustrated as in use on a canine;

FIG. 2 is a top plan view of the invention, a flexible web of the invention in a flat configuration;

FIG. 3 is a bottom plan view of the invention, the flexible web of the invention in the flat configuration;

FIG. 4 is a bottom plan view of an accessory of the invention, the accessory taking the form of a plush animal; and

FIG. 5 is a right-side elevational view of the invention, illustrated with the accessory fixed between a canine and the flexible web.

### DETAILED DESCRIPTION OF THE PREFERRED EMBODIMENT

Illustrative embodiments of the invention are described below. The following explanation provides specific details for a thorough understanding of and enabling description for these embodiments. One skilled in the art will understand that the invention may be practiced without such details. In other instances, well-known structures and functions have not been shown or described in detail to avoid unnecessarily obscuring the description of the embodiments.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively. Additionally, the words “herein,” “above,” “below” and words of similar import, when used in this application, shall refer to this application as a whole and not to any particular portions of this application. When the claims use the word “or” in reference to a list of two or more items, that word covers all of the following interpretations of the word: any of the items in the list, all of the items in the list and any combination of the items in the list.

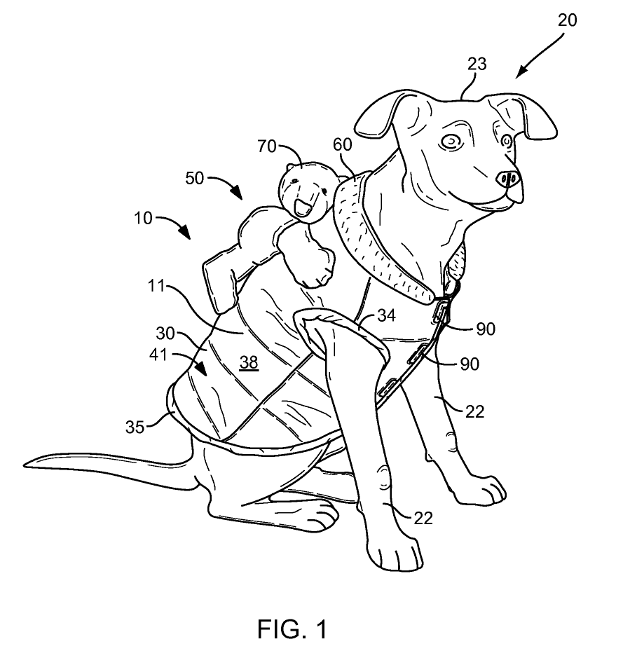

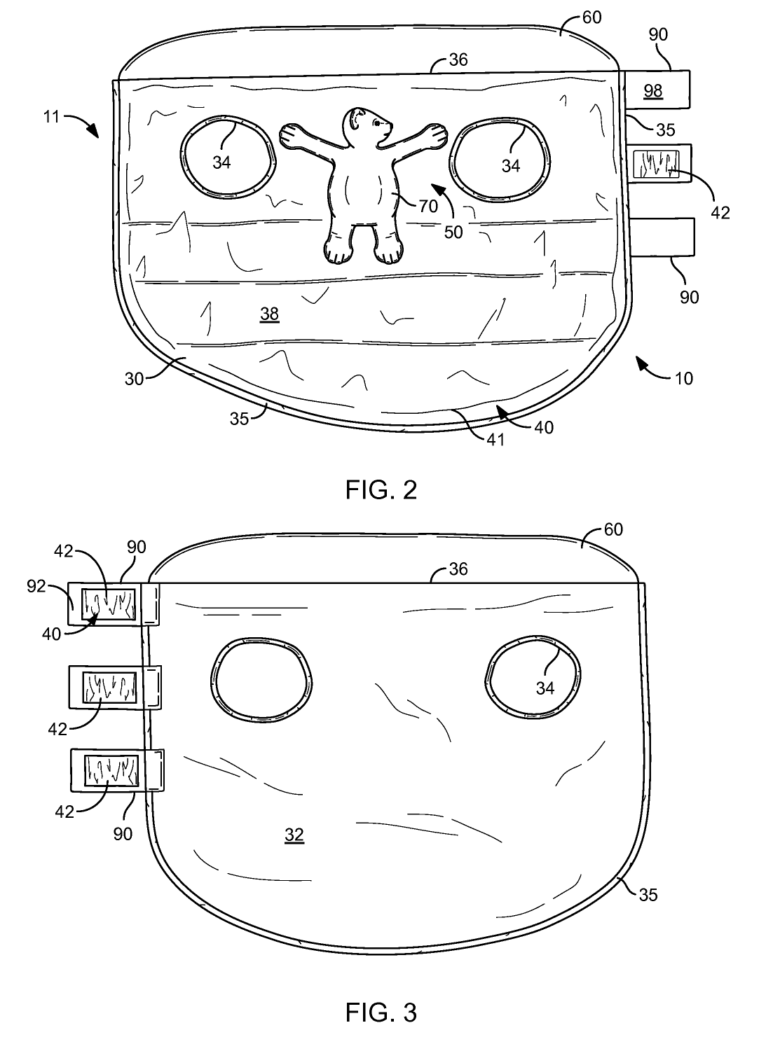

FIG. 1 illustrates a garment 10 for a mammal 20, such as a canine 23. A flexible web 30 has a first part 41 of a two-part fastening means 40 on an outer surface 38 thereof. The flexible web 30 includes at least one tab 90 at a peripheral edge 35 thereof (FIGS. 2 and 3). Each tab 90 includes a second part 42 of the two-part fastening means 40 on an inner surface 92 thereof, and each tab 90 may be made from an elastic flexible fabric, nylon sheet material, or the like.

One portion 36 of the peripheral edge 35 of the flexible web 30 may include a plush fabric material 60 (FIGS. 1-3), such as wool, padded silk, or the like. Such a plush fabric material 60 may be used where the peripheral edge 35 of the web 30 may rub uncomfortably while used on the mammal 20, such as near a canine's neck area, for example.

Preferably the two-part fastening means 40 is a hook-and-loop type fastening means. As such, the first part 41 may be the loop part of the hook-and-loop type fastening means 40, and the second part 42 may be the hook part of the hook and loop type fastening means 40, or vice versa. As such, the flexible web 30 may be made from a tricot material, for example, of cotton, wool, silk, rayon, nylon, or a combination of such fibers, resulting in a natural loop-type texture suitable for use as the first part 41 of the two-part fastening means 40. In such an embodiment, the first part 41 of the two-part fastening means 40 is integrally made with the flexible web 30. In such an embodiment, both the inner surface 32 and the outer surface 38 of the web 30 may include the natural loop-type texture suitable for use as the first part 41 of the two-part fastening means 40. As such, the tabs 90 may be fixed to the web 30 at a wide variety of locations, greatly increasing the flexibility of use of the garment 10 and the sizes of mammals 20 to which the garment 10 may be fixed.

Alternately, the two-part fastening means 40 may be other suitable fastening means. For example, two-part mechanical snaps (not shown) having a first female part and a second male part may be used. Likewise, a magnetic fastener comprising a magnetically attractive flexible sheet (not shown) may be fixed to the web 30, magnets (not shown) provided as the second-part of such a two-part fastening means 40. Alternately, adhesive fasteners (not shown) comprising a vinyl sheet first part and reusable adhesive tabs as the second part may be used. Clearly other types of suitable and well-known fastening means may be utilized as the two-part fastening means 40, such as hooks, catches, latches, buckles, zippers, clamps, ties, buttons, or the like.

An accessory 50 is selectively attachable to the web 30 and includes at least one of the second part 42 of the two-part fastening means 40. The accessory 50 may include a thermal pack 80 adapted to radiate heat through the flexible web 30 to the mammal 20 at the desired location thereon, or to lower the temperature of the mammal 20 proximate the desired location thereon. Such a thermal pack 80 may be a chemical thermal pack that, when activated, produces either an exothermic or endothermic reaction, for example. Alternately, such a thermal pack 80 may include a substance that when heated in a microwave, for example, radiates heat thereafter for an extended period of time. For example, the thermal pack 80 may comprise a cotton-lined bag filled with flaxseed and buckwheat, a polyester filling material, or the like. An aromatic substance 120 such as dried lavender, herbs, seeds, flowers, scented oils, scented fabrics, or the like, may also be included for producing a pleasant scent when heated. The thermal pack 80 may also include a material that, when frozen, absorbs heat thereafter for an extended period of time, such as Blue Ice brand products manufactured by Rubbermaid of Fairlawn, Ohio.

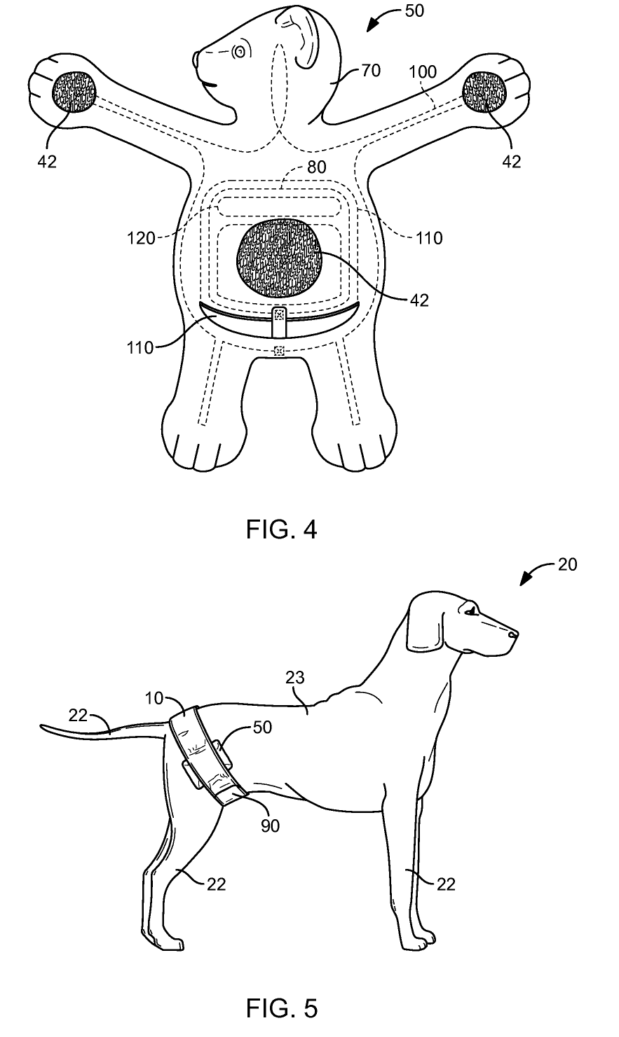

The accessory 50 may take the form of a plush animal 70, for example, which includes at least one selectively closable pocket 110 for containing the thermal pack 80, the aromatic substance 120, or other items such as water, food, treats, or the like (not shown). In such an embodiment, the plush animal 70 may further include a pliable endoskeleton therein, made from a pliable metal framework for example, such that the posturing of the plush animal 70 may be selectively configured. Preferably the second part 42 of the two-part fastening means 40, in such an embodiment, is fixed at least to paws, hands, or feet of the plush animal (FIG. 4) so that the plush toy 70 appears to be holding onto the mammal 20. Other types of accessories may be used, such as MP3 player carriers (not shown), travel pouches, canteen holders (not shown), and the like. Clearly multiple accessories 50 may be used simultaneously, in particular on larger mammals.

When used with mammals 20 having members 22 (FIGS. 1-3) such as legs, arms, tails, and heads, such as with canines, the flexible web 30 may further including at least one aperture 34 therethrough for receiving the member 22 of the mammal 20 therethrough. The web 30 may take the form of a jacket 11 for canines 23, for example, which includes two of the apertures 34 therethrough for the front legs of the canine 23. Clearly the garment 10 may be made in any size or shape as appropriate for each mammal 20. For example, the garment 10 may take the form of a wrap, scarf, band (FIG. 5), bandana, vest, shirt, pants, shorts, socks, undergarments, hat, visor, mask, headband, or the like. In one embodiment without apertures 34, each tab 90 may be made of any suitable length such that the garment 10 may wrap at least partially around the mammal 20 and be secured by each tab 90 to the mammal 20 (FIG. 5). The tabs 90 do not necessarily have to be fixed to the same peripheral edge 35 of the web 30, so that the tabs 90, which may also be referred to as “straps,” may be used to secure the web 30 around different portions of the mammal 20.

In one embodiment, each tab 90 further includes the first part 41 of the two-part fastening means 40 on an outer surface 98 thereof, and the web includes the first part 41 of the two-part fastening means 40 on the inner surface 32 thereof. In such an embodiment, the garment 10 is reversible, and can be applied to the mammal 20 either with the outer surface 38 facing away from the mammal 20, or with the inner surface 38 facing away from the mammal 20. The inner surface 32 and the outer surface 38 may be made from materials of different, perhaps contrasting colors, and as such the user may choose which surface 32,38 is displayed while in use.

In use, the flexible web 30 is wrapped around the mammal 20 and fixed thereto with the two-part fastening means 40. The accessory 50 may be attached to the outer surface 38 of the flexible web 30 at any desired location thereon, and, in the embodiment wherein the accessory 50 takes the form of a plush animal 70 with an inner pliable endoskeleton 100, the plush animal 70 may be selectively postured. In the embodiment wherein the first part 41 of the two-part fastening means 40 is included on the inner surface of the flexible web (FIG. 5), the accessory 50 may be fixed to the inner surface 32 of the web 30, between the web 30 and the mammal 20, such as to apply more direct heat or cold to an area of the mammal 20, for example.

While a particular form of the invention has been illustrated and described, it will be apparent that various modifications can be made without departing from the spirit and scope of the invention. For example, the garment 10 may be adapted for use with other mammals 20, such as humans, cats, farm animals, or the like. Accordingly, it is not intended that the invention be limited, except as by the appended claims.

The teachings provided herein can be applied to other systems, not necessarily the system described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions, and concepts of the various references described above to provide yet further embodiments of the invention.

These and other changes can be made to the invention in light of the above Detailed Description. While the above description details certain embodiments of the invention and describes the best mode contemplated, no matter how detailed the above appears in text, the invention can be practiced in many ways. Details of the system may vary considerably in its implementation details, while still being encompassed by the invention disclosed herein.

Particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific embodiments disclosed in the specification, unless the above Detailed Description section explicitly defines such terms. Accordingly, the actual scope of the invention encompasses not only the disclosed embodiments, but also all equivalent ways of practicing or implementing the invention.

The above detailed description of the embodiments of the invention is not intended to be exhaustive or to limit the invention to the precise form disclosed above or to the particular field of usage mentioned in this disclosure. While specific embodiments of, and examples for, the invention are described above for illustrative purposes, various equivalent modifications are possible within the scope of the invention, as those skilled in the relevant art will recognize. Also, the teachings of the invention provided herein can be applied to other systems, not necessarily the system described above. The elements and acts of the various embodiments described above can be combined to provide further embodiments.

All of the above patents and applications and other references, including any that may be listed in accompanying filing papers, are incorporated herein by reference. Aspects of the invention can be modified, if necessary, to employ the systems, functions, and concepts of the various references described above to provide yet further embodiments of the invention.

Changes can be made to the invention in light of the above “Detailed Description.” While the above description details certain embodiments of the invention and describes the best mode contemplated, no matter how detailed the above appears in text, the invention can be practiced in many ways. Therefore, implementation details may vary considerably while still being encompassed by the invention disclosed herein. As noted above, particular terminology used when describing certain features or aspects of the invention should not be taken to imply that the terminology is being redefined herein to be restricted to any specific characteristics, features, or aspects of the invention with which that terminology is associated.

In general, the terms used in the following claims should not be construed to limit the invention to the specific embodiments disclosed in the specification, unless the above Detailed Description section explicitly defines such terms. Accordingly, the actual scope of the invention encompasses not only the disclosed embodiments, but also all equivalent ways of practicing or implementing the invention under the claims.

While certain aspects of the invention are presented below in certain claim forms, the inventor contemplates the various aspects of the invention in any number of claim forms. Accordingly, the inventor reserves the right to add additional claims after filing the application to pursue such additional claim forms for other aspects of the invention.

What is claimed is:

1. A garment for a mammal comprising:
a flexible web having a first part of a two-part fastening means on an outer surface thereof, the flexible web including at least one tab at a peripheral edge thereof, each tab having a second part of the two-part fastening means on an inner surface thereof,
at least one accessory selectively attachable to the web, each accessory including at least one of the second part of the two-part fastening means;
whereby with the flexible web encircling the mammal and fixed thereto, the two-part fastening means holding the web on the mammal, each accessory may be attached to the outer surface of the flexible web at any desired location thereon.

2. The garment of claim 1 wherein the two-part fastening means is a hook-and-loop type fastening means, and wherein the first part of the two-part fastening means is a loop material and the second part of the two-part fastening means is a hook material.

3. The garment of claim 1 wherein the flexible web includes at least one aperture therethrough for receiving a member of the mammal therethrough.

4. The garment of claim 1 wherein at least one portion of the peripheral edge of the flexible web includes a plush fabric material.

5. The garment of claim 1 wherein the at least one accessory is in the form of a plush animal.

6. The garment of claim 1 wherein the accessory includes a thermal pack adapted to change the temperature of the mammal at the desired location thereon.

7. The garment of claim 6 wherein the thermal pack is adapted to be heated in a microwave.

8. The garment of claim 6 wherein the thermal pack is adapted to be chilled in a freezer.

9. The garment of claim 1 wherein the flexible web further includes the first part of the two-part fastening means on an inner surface thereof, and wherein each tab includes the second part of the two-part fastening means on an outer surface thereof, whereby the garment is reversible and each accessory may be placed on either the inner or outer surface of the flexible web.

10. The garment of claim 9 wherein the inner surface of the garment contrasts visually with the outer surface of the garment.

11. The garment of claim 5 wherein the plush animal includes a pliable endoskeleton therein, such that the posturing of the plush animal may be selectively configured.

12. The garment of claim 3 wherein the flexible web takes the form of a jacket for canines, and includes two of the apertures therethrough for the front legs of the canine.

13. The garment of claim 1 wherein the accessory includes a selectively closable pocket.

14. The garment of claim 1 wherein the accessory includes an aromatic substance.
